(19) United States
(12) Patent Application Publication
(10) Pub. No.: US 2026/0335875 A1
(43) Pub. Date: Oct. 29, 2026

(54) COPPER-CONTAINING HYDROGENATION CATALYSTS FOR THE HYDROGENOLYSIS OF ESTERS

(71) Applicant: Johnson Matthey Davy Technologies Limited, London (GB)

(72) Inventor: Stuart Michael SMALL, Billingham Cleveland (GB)

(21) Appl. No.: 19/106,064

(22) PCT Filed: Jun. 29, 2023
(86) PCT No.: PCT/GB2023/051692

§ 371 Date: Feb. 24, 2025

(30) Foreign Application Priority Data
Sep. 23, 2022 (GB) 2213961.2

(51) Int. Cl. B01J 35/51; B01J 35/30; B01J 37/02; B01J 37/16; C07C 51/09

(52) U.S. Cl. CPC B01J 35/51; B01J 35/392; B01J 37/0201; B01J 37/0236; B01J 37/16; C07C 51/09; C07C 2521/04; C07C 2523/80

(57) ABSTRACT

The specification describes a process for the hydrogenolysis of an ester substrate, comprising the step of carrying out hydrogenolysis of the ester substrate in the presence of a heterogeneous catalyst, wherein the catalyst comprises: 5-20 wt % copper and 5-12 wt % zinc; on an alumina support; wherein the total content of elements other than Cu, Zn, Al and O is ≤1 wt %. Also described are oxidic catalyst for the hydrogenolysis of an ester substrate, wherein the catalyst comprises: 5-20 wt % copper and 5-12 wt % zinc; on an alumina support; wherein the catalyst has a copper surface area of 10-20 m²/mL_{cat}; and wherein the total content of elements other than Cu, Zn, Al and O is ≤1 wt %. Also described is a method of preparing such oxidic catalysts.

### FIELD OF THE INVENTION

The present invention relates to copper-containing hydrogenation catalysts and their use in the hydrogenolysis of esters.

### BACKGROUND

Catalytic hydrogenolysis of esters is a commercially important reaction. The hydrogenolysis reaction is:

RCOOR′+2H₂→RCH₂OH+R′OH

Fatty alcohols are useful feedstocks, in particular for the production of surfactants and detergents. One process for the production of fatty alcohols is through the hydrogenolysis of acid methyl esters (FAMEs) which are usually derived from triglycerides found in animal fats and vegetable oils.

The hydrogenolysis or hydrogenation of FAME feedstocks using catalysts containing copper and chromium has been described in the prior art, for example in U.S. Pat. No. 2,091,800. WO91/04789 describes an acid-resistant copper chromite catalyst material containing promoter metal compounds as well as colloidal silicic acid, and a process for their production and use for direct fixed-bed hydration of fatty acids to produce fatty alcohols of appropriate chain-length. Due to the toxicity of some chromium species, there has been much research into chromium-free catalysts for ester hydrogenolysis.

JPH1045645A describes a process for the production of cyclohexanedimethanol (CHDM) by a two-stage process. The first stage involves hydrogenating a dialkyl terephthalate in the presence of a noble metal-based nuclear hydrogenation catalyst to produce 1,4-cyclohexanedicarboxylic dialkyl ester. The second stage involves hydrogenolysis of the 1,4-cyclohexanedicarboxylic dialkyl ester in the presence of a copper-zinc-alumina-based catalyst. A preferred catalyst for the second stage comprises, in oxidic form, 30-70 wt % copper oxide, 20-60 wt % zinc oxide and 3-20 wt % alumina.

The article “Cu—Zn/Al₂O₃ Catalyst for the Hydrogenation of Esters to Alcohols” (Chin J Catal, 2010, 31: 769-775) describes a catalyst with a molar ratio of copper:zinc:alumina of 30:40:30 which is produced by a high speed collision co-precipitation method. The catalyst contained approximately 51 wt % copper, 10 wt % zinc and 7 wt % aluminium after calcination. The catalyst was tested for its performance in the hydrogenolysis of natural palm oil which has first been transesterified with methanol.

WO2005/070537A1 describes chromium-free catalysts comprising copper and at least one second metal which are obtainable through a method involving addition of a solution comprising Cu ions and ions of at least one other second metal to an inert carrier, optional drying, calcining and reduction of at least part of the oxidic copper. The examples use silica, magnesia, zirconia or titania as the support. The catalysts may be used for a number of duties, including the hydrogenation of fatty acids, fatty esters, esters and diesters to fatty alcohols, alcohols and dialcohols respectively.

WO2016/059431A1 describes catalysts comprising copper, zinc and a silica-alumina material that is not a zeolite. In the examples catalysts are prepared by precipitating copper nitrate and zinc nitrate with sodium carbonate in the presence of a silica-alumina support.

The catalysts are tested for their activity in the hydrogenolysis of methyl laurate.

WO2016/154514A1 describes catalysts comprising a mixed metal oxide comprising copper and at least one of manganese, zinc, nickel or cobalt; an alumina; silica; and calcium. The catalysts may be used for a number of duties, including the hydrogenolysis of FAMEs and free fatty acids.

CN1036576567B describes hydrogenation catalysts comprising 3-10 mass % boron oxide, 6-20 mass % zinc oxide, 4-12 mass % copper, on a carrier of gamma alumina with a specific surface area of 180-400 m²/g and a pore volume of 0.2-0.8 cm³/g. The catalysts may be used for a variety of duties including the hydrogenation of FAMEs.

CN105363454B describes hydrogenation catalysts comprising: (a) 10-50 wt % copper or its oxides; (b) 0-20 wt % of a mixture of ZnO and CdO; (c) ≤5 wt % of a mixture of P₂O₅ and Bi₂O₃; and (d) 40-70 wt % of a carrier selected from at least one of silica, alumina, and a molecular sieve, on the condition that the amount of component (c) is not zero. Comparative catalyst C1 is 10CuO-15ZnO-75Al₂O₃ and is prepared by co-impregnation of an alumina carrier with a solution containing copper nitrate trihydrate and zinc nitrate hexahydrate. The catalysts may be used for a variety of duties including the hydrogenation of esters.

There is a need for further, improved, ester hydrogenolysis catalysts. Ideally an ester hydrogenolysis catalyst should satisfy the following properties. Firstly, the catalyst should have high activity for the conversion of the ester substrate. Secondly, the catalyst should retain high activity for the conversion of the ester substrate, so that the catalyst does not need to replaced too frequently. Thirdly, the catalyst should have high selectivity for the desired ester hydrogenolysis reaction and produce low levels of by-products. Fourthly, the catalyst should have high crush strength and retain high crush strength during the course of the reaction. The reason for this is that if the crush strength is too low, the catalyst may disintegrate in the reactor resulting in an increase in pressure drop through the reactor, which can cause blockages and requires premature catalyst discharge.

The present invention relates to a catalyst having a good balance of the above properties, and a process of using the catalyst in the hydrogenolysis of esters.

### DESCRIPTION OF THE FIGURES

FIG. 1 is a plot showing substrate conversion (methyl laurate) against copper surface area (m²/mL_{cat})

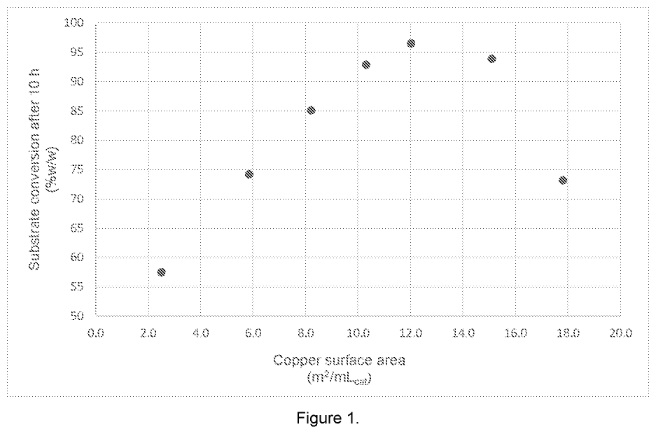

### SUMMARY OF INVENTION

The present inventors have now found that a catalyst containing copper and zinc on an alumina support provides a catalyst having a good balance between: high activity for ester hydrogenation, retention of activity, low by-product production and good retention of crush strength.

In a first aspect the invention relates to a process for the hydrogenolysis of an ester substrate, comprising the step of carrying out hydrogenolysis of the ester substrate in the presence of a heterogeneous catalyst, wherein the catalyst comprises:
5-20 wt % copper; and
5-12 wt % zinc;
on an alumina support;
wherein the total content of elements other than Cu, Zn, Al and O is ≤1 wt %.

The copper and zinc contents are measured on the oxidic catalyst by the method described in the examples section. The term “oxidic catalyst” means a catalyst in which substantially all of the Zn and Cu are present as oxides. The catalyst used in the process is in reduced form and is preferably prepared by reducing an oxidic catalyst as defined in the second aspect of the invention.

Once installed in a reactor the oxidic catalyst is typically activated in situ by reducing some or all of the CuO to Cu metal using a feed containing H₂. However, the ZnO and alumina are not reduced. The activation may be carried out in the gas phase or the liquid phase. The result is a catalyst in which some or all of the Cu is present as crystallites of Cu(0). Such catalyst is referred to as a “reduced catalyst” herein. An exemplary method for the liquid phase activation of a catalyst is described in WO2020/053555 (Johnson Matthey Davy Technologies Limited).

In preferred embodiments the substrate is an ester of a saturated or unsaturated fatty acid.

In a second aspect the invention relates to an oxidic catalyst for the hydrogenolysis of an ester substrate, wherein the catalyst comprises:
5-20 wt % copper; and
5-12 wt % zinc;
on an alumina support;
wherein the catalyst has a copper surface area of 10-20 m²/mL_{cat};
wherein the total content of elements other than Cu, Zn, Al and O is ≤1 wt %.

The copper content, zinc content and copper surface area are measured on the oxidic catalyst by the method described in the examples section.

In a third aspect the invention relates to a method for preparing an oxidic catalyst according to the second aspect, comprising the steps of:
(i) impregnating an alumina support with an impregnation solution comprising copper carbonate and zinc carbonate;
(ii) drying the product of step (i); and
(iii) calcining the product of step (ii).

### DETAILED DESCRIPTION

Any sub-headings are for convenience only and are not intended to limit the invention.

Any aspect described as being preferred in connection with the catalyst also applies to the catalyst used in the process of the first aspect or prepared by the method of the third aspect.

### Catalyst

The catalyst comprises 5-20 wt % copper. This loading of copper is lower than many known precipitated copper catalysts. A low content of copper is preferred from a cost perspective. A preferred copper content is 5-12 wt % as this gives a good balance between activity and cost.

The catalyst comprises 5-12 wt % zinc. The inclusion of zinc not only improves the catalyst activity in absolute terms, but also reduces the drop-off in catalyst activity over time, and decreases the levels of by-products produced. High zinc contents, namely above 12 wt %, whilst still providing a more active and selective catalyst compared to a copper on alumina catalyst, lead to a decrease in catalyst activity.

The total content of elements other than Cu, Zn, Al and O in the catalyst is ≤1 wt %. The content of elements can be determined by inductively coupled plasma mass spectrometry (ICP) or by X-ray fluorescence (XRF).

Whilst the contents copper, zinc and alumina will not be identical between the oxidic form and reduced form of the catalyst, given the relatively low levels of Cu and Zn in the catalysts of the present invention, they will be similar. The values of Cu, Zn and “total content of elements other than Cu, Zn, Al and O” therefore apply to both the oxidic catalyst and the reduced catalyst.

In a preferred embodiment the catalyst has a copper surface area of 10-20 m²/mL_{cat}. Copper surface area is measured by the procedure in the examples section. Whilst this surface area per unit volume is lower than some known precipitated catalysts, it is higher than many known catalysts of copper on an alumina support. A supported alumina catalyst containing copper and zinc, with a copper surface area per unit volume within this range has a high activity for the hydrogenolysis of esters. In a preferred embodiment the catalyst has a copper surface area of 11-17 m²/mL_{cat}.

The copper and zinc are supported on an alumina support. As used herein, the term “alumina” is not intended to encompass silica-aluminas. The silicon content of the alumina used herein is ≤5 wt %, preferably ≤2 wt % such as ≤1 wt %.

Alumina takes on various different forms depending on the temperature to which it has been calcined. The support is preferably α-alumina, γ-alumina or 6-alumina, or a mixture thereof. The alumina may be a single form or a mixture of different forms. In a preferred embodiment the alumina support is γ-alumina.

It is preferred that the catalyst is in the form of granules, spheres or multi-lobe shapes such as trilobes. Such catalysts can be prepared by impregnating an alumina support in the form of granules, spheres or multi-lobe shapes such as trilobes. It is preferred that the support (and the catalyst) is in the form of trilobes.

### Manufacture of the Catalyst

The catalysts described herein may be prepared by impregnation of an alumina support. The catalysts may be manufactured by co-impregnation using a solution containing a copper salt and a zinc salt, or by sequential impregnation using a solution containing a copper salt and a separate solution containing a zinc salt (in any order). The co-impregnation method is preferred.

Impregnation techniques will be well known to those skilled in the art. Typically, an impregnation involves preparing an impregnation solution comprising the salt(s) to be impregnated and adding a volume of impregnation solution to the support, in which the volume of impregnation solution is approximately equal to the absorption volume of the support.

A preferred copper salt is copper carbonate, which may be provided as a solution of copper carbonate in ammonium hydroxide for impregnation.

A preferred zinc salt is zinc carbonate, which may be provided as a solution of zinc carbonate in ammonium hydroxide for impregnation.

It is particularly preferred that the copper salt and zinc salt are provided in the same solution and that the metals are applied on the support using a co-impregnation method.

Following the impregnation step (i) the product is dried (step (ii)). If desired steps (i) and (ii) may be repeated two or more times; this may be desirable if loadings of Cu and Zn towards the higher end of the range are required.

A calcination step (step (iii)) is carried out once all of the required metal(s) have been impregnated onto the support. Typical calcination conditions are 200-400 °C for 4 h±2 h.

The skilled person will readily be able to determine calcination conditions, which may vary depending on scale and equipment used.

### Hydrogenolysis Process

The catalysts described herein are suitable for the hydrogenolysis of ester substrates to the corresponding acid and alcohol. Preferred substrates are the esters of C6-C22 fatty acids, such as the esters of C8-C20 fatty acids. The fatty acid component of the ester may be saturated or unsaturated and may be linear or branched. The ester is preferably a methyl ester.

### EXAMPLES

### Analytical Procedures

### Tapped Bulk Density

~40 g of catalyst was weighed into a 3 cm diameter measuring cylinder and tapped 2000 times using a jolt bulk density meter, the volume of the catalyst was then recorded. The density was calculated by dividing the mass charged with the volume recorded.

### Copper Surface Area

Copper surface areas were determined using reverse frontal chromatography as follows: oxidic catalyst particles were crushed and sieved to a particle size of 0.6 to 1.0 mm. About 2.0 g of the crushed material was weighed into a stainless-steel tube and heated to 68 °C and purged with helium for 2 minutes. Then, the catalyst was reduced by heating it in a flow of 5% vol hydrogen in helium, at 4 °C/min up to 230 °C and holding at this temperature for 30 minutes until fully reduced. The reduced catalyst was cooled to 68 °C under helium. The reduced catalyst then had a 2.5% vol N₂O in helium gas mixture passed over the catalyst. The evolved gases were passed through a gas chromatograph and the N₂ evolution measured.

The copper surface area per gram of charged catalyst was calculated (m²/g_{cat}) and then multiplied by the tapped bulk density (g_{cat}/mL) to give copper surface area per mL (m²/mL_{cat}).

### Strength

Crush strength was measured using an Engineering systems CT6 instrument. A crush speed of 22 mm/min was used with a 50 kg load cell. 20 catalyst particles were analysed radially and the average value calculated.

### Catalyst Testing Procedure

10 mL of catalyst, based on the tapped bulk density, was charged to a stainless-steel reactor in the presence of 45 g fine (0.1-0.3 mm) silicon carbide. Silicon carbide was also added above and below the catalyst. The catalysts were activated in a 0.5 L/min flow of 5% hydrogen in nitrogen at 0.6 barg by heating to 240 °C at a rate of 1 °C/min and holding at these conditions for four hours before reducing the reactor to 215 °C and commencing the catalyst test program.

The gas was changed to 100% hydrogen and metered into the rig at 2.8 L/min. Methyl laurate was introduced at a flow rate of 0.375 mL/min once a reactor pressure of 250 barg was achieved. This equates to a 82:1 H₂:ester molar ratio and an LHSV of 2.25 hr⁻¹. The reactor oil jacket temperature was maintained at 215 °C. These conditions were held for 168 hours with online GC analysis completed throughout. The flow of hydrogen and methyl laurate was then reduced to 1.2 L/min and 0.167 mL/min respectively resulting in an LHSV of 1.0 hr⁻¹ but with retention of the H₂:ester ratio. The temperature of the oil jacket was increased and allowed to stabilise at the following temperatures for ~5 hours with online GC analysis of the product completed at each temperature: 215, 220 and 225 °C.

The liquid flow was then isolated with all other conditions maintained for ~2 hours to purge the system/catalyst of liquid. The reactors were then cooled to room temperature, purged with 5% hydrogen in nitrogen before being vacuum discharged. The discharged catalyst was analysed for strength and compared to the fresh.

### GC Analysis

Online GC analysis was completed using a Bruker 456 GC to determine concentrations of the following components:

Methanol, Octene, Octane, Hexanol, Nonane, Heptanol, Decene, Decane, Octanal, Octyl methyl ether, Octanol, 2-Nonanone, 2-Nonanol, Methyl octanoate, Octanoic acid, Nonanol, Dodecene, Dodecane, Decanal, Decyl methyl ether, Decanol, 2-Undecanone, 2-Undecanol, Methyl decanoate, Decanoic acid, Undecanol, Tetradecene, Tetradecane, Dodecanal, Dodecyl methyl ether, Dodecanol, Pentadecane, Methyl dodecanoate, Dodecanoic acid, C16 wax ether, C16 wax ester, C18 wax ether, C18 wax ester, C20 wax ether, C20 wax ester, C22 wax ether, C22 wax ester, C24 wax ether, C24 wax ester.

Each component was calibrated for using an external standard; any unknowns were estimated using the response factor for dodecanol.

### GC Parameters

Column: CP-Sil 8 CB 50 m 0.32 mm 1.2 um
Injector: 250 °C
Split 30:1
Column flow 2.0 ml/min (constant flow)
Oven: 50 °C for 0.5 min, 220 °C @10 °C/min, 300 °C @5 °C/min with a 15 min hold
Detector: 325 °C.

### Example 1 (Comparative)—Cu and Zn Precipitated Catalyst (C1)

C1 was prepared following the procedure described in Example 1 of WO2016/059431A1.

### Example 2 (Comparative)—Cu and Zn Precipitated Catalyst (C2)

C2 was a commercially available precipitated catalyst PRICAT™ CZ 40/18T (Johnson Matthey).

### Example 3 (Comparative)—Cu on Alumina Prepared by Impregnation (C3)

C3 was prepared following a procedure adapted from WO99/51340A1.

An impregnation solution was prepared by dissolving 211.39 g of ammonium carbonate, 253.57 g of basic copper carbonate in 800 mL of ammonium hydroxide. 50 g of 2.5 mm diameter gamma alumina trilobes were impregnated with the solution until all pores were filled. This was dried at 120 °C for four hours before being impregnated a second time. Once all of the pores were filled with the impregnation solution the sample was dried at 120 °C for four hours and then calcined at 300 °C for four hours. The sample was analysed to contain 14.5 wt. % Cu and 41.1 wt. % Al by XRF and have a copper surface area of 5.8 m²/mL_{cat}.

### Example 4—Cu and Zn on Alumina Prepared by Impregnation (E4)

The catalyst was prepared in the same manner as C3 with an impregnation solution consisting of 317.09 g of ammonium carbonate, 194.20 g of basic copper carbonate and 189.47 g of basic zinc carbonate in 1200 mL of ammonium hydroxide. The sample was analysed to contain 8.5 wt. % Cu, 8.4 wt. % Zn and 37.6 wt. % Al by XRF and have a copper surface area of 12.0 m²/mL_{cat}.

### Example 5 (Comparative)—Cu and Zn on Zinc Oxide Prepared by Impregnation (C5)

The catalyst was prepared following the same procedure as E4 but using KATALCO™ 32-4, a 3-4 mm zinc oxide sphere, instead of the alumna trilobes. The sample was analysed to contain 7.0 wt. % Cu, 60.2 wt. % Zn and 1.4 wt. % Al by XRF and have a copper surface area of 8.2 m²/mL_{cat}.

### Example 6—Cu and Zn on Alumina Prepared by Impregnation (E6)

The catalyst was prepared in the same manner as C3 with an impregnation solution consisting of 52.85 g of ammonium carbonate, 26.54 g of basic copper carbonate and 37.26 g of basic zinc carbonate in 200 mL of ammonium hydroxide. Three impregnations were applied instead of two. The sample was analysed to contain 9.0 wt. % Cu, 13.0 wt. % Zn and 35.3 wt. % Al by XRF and have a copper surface area of 10.3 m²/mL_{cat}.

### Example 7 (Comparative)—Cu and Zn on Silica Prepared by Impregnation (C7)

The catalyst was prepared in the same manner as C3 but using CARiACT Q-20C, a 4 mm silica dioxide sphere supplied by Fuji Silysia, instead of the alumna trilobes. The sample was analysed to contain 8.3 wt. % Cu, 8.7 wt. % Zn and 34.3 wt. % Si by XRF and have a copper surface area of 2.5 m²/mL_{cat}.

| | C1 | C2 | C3 | E4 | C5 | E6 | C7 |
|---|---|---|---|---|---|---|---|
| Preparation method | Precipitation | Precipitation | Impregnation | Impregnation | Impregnation | Impregnation | Impregnation |
| Support | N/A | N/A | γ-Al₂O₃ | γ-Al₂O₃ | ZnO | γ-Al₂O₃ | SiO₂ |
| Cu (wt %) | 33.8 | 37.0 | 14.5 | 8.5 | 7.0 | 9.0 | 8.3 |
| Zn (wt %) | 36.9 | 23.6 | — | 8.4 | 60.2 | 13.0 | 8.7 |
| Al (wt %) | 3.5 | 5.1 | 41.1 | 37.6 | 1.4 | 35.3 | — |
| Si (wt %) | 0.3 | — | — | — | — | — | 34.3 |
| Cu SA | 8.2 | 22.0 | 8.8 | 15.9 | 5.9 | 12.7 | 3.9 |
| (m²/g_{cat}) | | | | | | | |
| Cu SA | 15.1 | 17.8 | 5.8 | 12.0 | 8.2 | 10.3 | 2.5 |
| (m²/mL_{cat}) | | | | | | | |
| Conversion after | 93.9 | 73.2 | 74.2 | 96.5 | 85.1 | 92.9 | 57.5 |
| 10 h (w/w) | | | | | | | |
| Conversion after | 84.0 | 67.4 | 31.1 | 93.6 | 78.8 | 85.5 | N/A* |
| 150 h (w/w) | [89.5] | [92.1] | [41.9] | [97.0] | [92.6] | [92.0] | |
| [% of 10 h value] | | | | | | | |
| By-products after | 0.23 | 0.10 | 1.35 | 0.41 | 0.10 | 0.31 | 0.14 |
| 10 h (wt %) | | | | | | | |
| Fresh strength | 9.4 | 14.8 | 9.9 | 13.1 | 4.8 | 12.3 | 28.6 |
| (kgF) | | | | | | | |
| Discharged | 5.3 | 4.1 | 9.6 | 8.1 | 1.9 | 10.1 | 25.2 |
| strength (kgF) | [56] | [27] | [97] | [62] | [40] | [82] | [88] |
| [% of fresh | | | | | | | |
| strength] | | | | | | | |

*Not measured. The catalyst had low activity and conversion had dropped to 52.1% after 23 hours.

E4 and E6 showed an excellent balance between:
(1) activity for hydrogenolysis;
(2) prolonged activity, as measured by the absolute value of conversion after 150 h and % of the conversion at 10 h;
(3) level of by-products, especially when compared to an impregnated catalyst on alumina but without Zn addition (C3);
(4) good absolute strength and strength retention.

### Example 8 (Comparative)—Cu on Alumina Prepared by Impregnation Using Copper Nitrate (C8)

An impregnation solution was prepared by dissolving 27.9 g of copper nitrate hemipentahydrate in deionised water to a volume of 26.6 mL. 50 g of 2.5 mm diameter delta/theta alumina trilobes were impregnated with the solution until all pores were filled. This was dried at 120 °C for four hours and then calcined at 300 °C for four hours. The sample was analysed to contain 15.8 wt. % Cu by XRF and have a copper surface area of 4.4 m²/mL_{cat}.

### Example 9 (Comparative)—Cu on Alumina Prepared by Impregnation Using Copper Carbonate (C9)

The catalyst was prepared following the same procedure as E4 but using 2.5 mm diameter delta/theta alumna trilobes, instead of gamma alumina. The sample was analysed to contain 17.8 wt. % Cu by XRF and have a copper surface area of 5.4 m²/mL_{cat}. The Cu loading of C9 was ~13% higher than C8 but the Cu surface area was ~23% higher. This demonstrates that the use of copper carbonate instead of copper nitrate as the salt for the impregnation may be used to produce catalysts with comparably higher copper surface area.

1. A process for the hydrogenolysis of an ester substrate, comprising the step of carrying out hydrogenolysis of the ester substrate in the presence of a heterogeneous catalyst, wherein the catalyst comprises:
5-20 wt % copper; and
5-12 wt % zinc;
on an alumina support;
wherein the total content of elements other than Cu, Zn, Al and O is ≤1 wt %.

2. A process according to claim 1, wherein the catalyst comprises 5-12 wt % copper.

3. A process according to claim 1, wherein the alumina support is α-alumina, γ-alumina or θ-alumina or a mixture thereof.

4. A process according to claim 1, wherein the alumina support is in the form of granules, spheres or multi-lobe shapes.

5. A process according to claim 1, wherein the alumina support is in the form of trilobes.

6. A process according to claim 1, wherein the ester substrate is an ester of a fatty acid.

7. A process according to claim 1, wherein the ester substrate is an ester of a C6-C22 fatty acid.

8. A process according to claim 1, wherein the ester substrate is a methyl ester.

9. A process according to claim 1, wherein the catalyst is prepared by reducing an oxidic catalyst for the hydrogenolysis of an ester substrate, wherein the catalyst comprises:
5-20 wt % copper; and
5-12 wt % zinc;
on an alumina support;
wherein the catalyst has a copper surface area of 10-20 m²/mL_{cat}; and
wherein the total content of elements other than Cu, Zn, Al and O is ≤1 wt % and where included is an oxidic catalyst for the hydrogenolysis of an ester substrate, wherein the catalyst comprises:
5-20 wt % copper; and
5-12 wt % zinc;
on an alumina support;
wherein the catalyst has a copper surface area of 10-20 m²/mL_{cat}; and
wherein the total content of elements other than Cu, Zn, Al and O is ≤1 wt %.

10. An oxidic catalyst for the hydrogenolysis of an ester substrate, wherein the catalyst comprises:
5-20 wt % copper; and
5-12 wt % zinc;
on an alumina support;
wherein the catalyst has a copper surface area of 10-20 m²/mL_{cat}; and
wherein the total content of elements other than Cu, Zn, Al and O is ≤1 wt %.

11. An oxidic catalyst according to claim 10, wherein the catalyst comprises 5-12 wt % copper.

12. An oxidic catalyst according to claim 10, wherein the alumina support is α-alumina, γ-alumina or θ-alumina or a mixture thereof.

13. An oxidic catalyst according to claim 10, wherein the alumina support is in the form of granules, spheres or multi-lobe shapes.

14. An oxidic catalyst according to claim 10, wherein the alumina support is in the form of trilobes.

15. A method for preparing an oxidic catalyst according to claim 10, comprising the steps of:
(i) impregnating an alumina support with an impregnation solution comprising copper carbonate and zinc carbonate;
(ii) drying the product of step (i); and
(iii) calcining the product of step (ii).
